(19) United States
(12) Patent Application Publication
(10) Pub. No.: US 2020/0015681 A1
(43) Pub. Date: Jan. 16, 2020

(54) SMART PHONE-CONTROLLED AUDIO AND VIDEO MONITORING DEVICE FOR DETECTING PHYSIOLOGICAL INFORMATION

(71) Applicant: ZHENG LI, Shenzhen (CN)

(72) Inventor: ZHENG LI, Shenzhen (CN)

(21) Appl. No.: 16/056,588

(22) Filed: Aug. 7, 2018

(30) Foreign Application Priority Data
Jul. 12, 2018 (CN) 2018107636425

(51) Int. Cl. A61B 5/00; H04N 7/025; A61B 5/0205

(52) U.S. Cl. CPC A61B 5/0013; H04N 7/025; H04W 88/06; A61B 5/681; A61B 5/6898; A61B 5/02055

(57) ABSTRACT

An audio and video monitoring device for detecting physiological information which can be controlled by a terminal device is disclosed. The monitoring device includes a vital signs monitor and a network camera. The vital signs monitor includes a photoelectric sensor, a motion sensor, a body temperature sensor, a GPS sensor, a monitor MCU, a wireless transmitting module, a battery, and an A/D converting module. The photoelectric sensor includes a photoelectric heart rate sensor, a photoelectric blood oxygen sensor, and a blood pressure sensor. The monitoring device can transmit the physiological characteristics signal and audio and video signal synchronously to the remote terminal device, and it is safe to the special users, such as the babies or the elders.

### FIELD OF THE INVENTION

The present invention relates generally to electrical and electronic hardware, computer software, wired and wireless network communications, and computing devices, more specifically, to an A/V monitoring device and a monitoring system for detecting physiological characteristics which can be controlled by a smart phone.

### BACKGROUND OF THE INVENTION

With advances in technology, various types of physiological information such as respiration rate, heart rate, blood pressure value and so forth are no longer limited to be measured by professional instruments. Locations where measurements may be taken also do not have to cope with large-scale instruments by only being set at medical institutions or other indoor environments. Currently, there are already many wearable devices targeting health management on the market. These type of devices are heavy and is hard to wear, and many devices are used to detect a plurality of physiological information measuring functions and to record various types of physiological information of a user.

### SUMMARY OF THE INVENTION

The technical problem to be solved by the present invention is to provide an audio and video monitoring device for physiological information which can be controlled by a smart phone.

In order to solve the above technical problem, one embodiment of the invention provides the following technical solution:

One embodiment of the invention provides a monitoring device for detecting physiological information which can be controlled by a terminal device. The monitoring device includes a vital signs monitor and a network camera. The vital signs monitor includes a photoelectric sensor, a motion sensor, a body temperature sensor, a GPS sensor, a monitor MCU, a wireless transmitting module, a battery, and an A/D converting module. The photoelectric sensor includes a photoelectric heart rate sensor, a photoelectric blood oxygen sensor, and a blood pressure sensor. The photoelectric heart rate sensor, the photoelectric blood oxygen sensor, the blood pressure sensor, the motion sensor, the body temperature sensor and the GPS sensor are coupled to the A/D converting module. The A/D converting module is coupled to the monitor MCU. The monitor MCU wirelessly is connected to the network camera. The network camera includes a Wi-Fi module, a mobile communication module, a RJ45 module, a data receiving module, a camera MCU and an A/V module. The data receiving module is coupled to the wireless transmitting module. The data receiving module and the A/V module are coupled to the camera MCU. The camera MCU is coupled to the router through the Wi-Fi module or the RJ45 module. The router is coupled to the server. The camera MCU is coupled to the router through the mobile communication module. The monitor MCU is electrically coupled to the network camera through the wireless transmitting module.

As a preferred technical solution of the embodiment of the invention, at least two of the vital signs monitor, the network camera, the router, the server and the terminal device have separate communicating protocol.

As a preferred technical solution of the embodiment of the invention, the motion sensor is a three-axis acceleration sensor.

As a preferred technical solution of the embodiment of the invention, the vital signs monitor and the network camera are wirelessly connected, and a communication protocol of the vital signs monitor and the network camera is selected from but not limited to the Bluetooth, the Microwave, the Zigbee, or the WI-FI.

As a preferred technical solution of the embodiment of the invention, the camera MCU comprises a codec that could re-code data from the vital signs monitor and the A/V signal, and could send the data to a smart phone.

As a preferred technical solution of the embodiment of the invention, the vital signs monitor is selected from a wrist band, an ankle band and a necklet, and the terminal device is a smart phone or a band.

### BRIEF DESCRIPTION OF THE DRAWINGS

The drawings are used for providing further understanding of the present invention, constitute a part of the description and are used for explaining the present invention together with the embodiment of the present invention, but do not constitute a limitation to the present invention.

In the drawings:

FIG. 1 is a schematic view of one embodiment of a monitoring device.

FIG. 2 is another schematic view of the monitoring device of FIG. 1.

FIG. 3 is a block view of the monitoring device of FIG. 1.

FIG. 4 is a schematic view of the monitoring device of FIG. 1 with communication protocols.

### DETAILED DESCRIPTION OF THE PREFERRED EMBODIMENT

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure.

Several definitions that apply throughout this disclosure will now be presented.

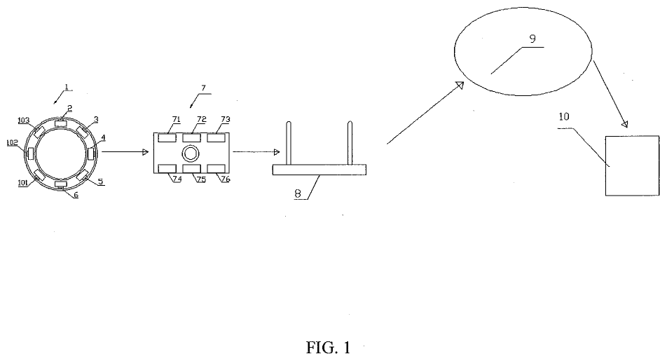

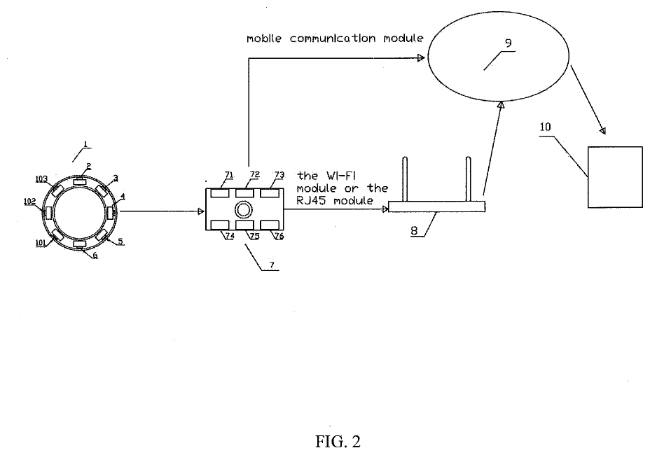

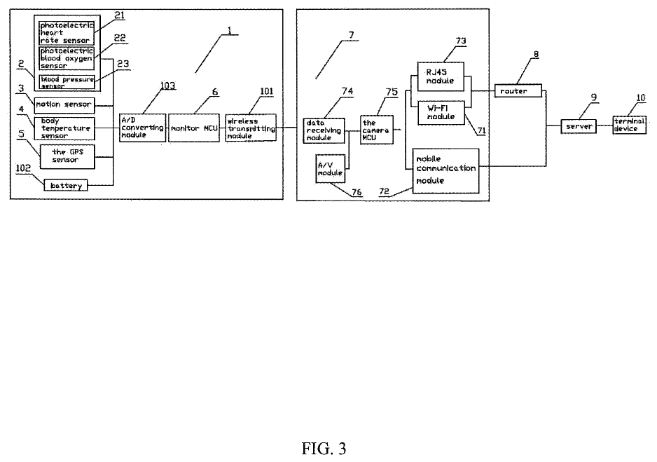

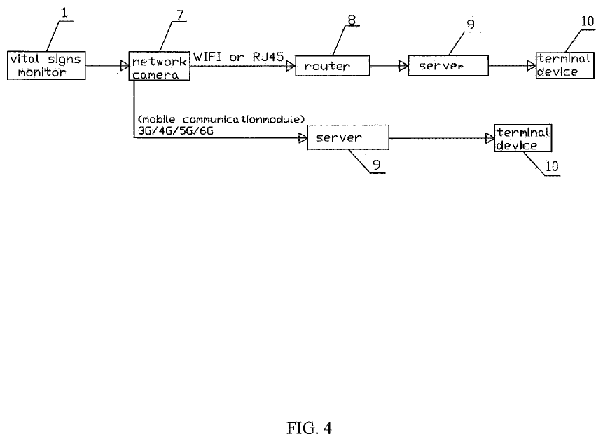

As shown through FIG. 1 to FIG. 4, in one embodiment, a monitoring device for physiological information is disclosed which can be controlled and monitored by a remote smart phone. The monitoring device includes a vital signs monitor 1 and a network camera 7 coupled to the vital signs monitor 1. A monitoring system may include a monitoring device and the smart phone. The vital signs monitor 1 includes a photoelectric sensor 2, a motion sensor 3, a body temperature sensor 4, a GPS (Global Position System) sensor 5, a monitor MCU (Micro-programmed Control Unit) 6, a wireless transmitting module 101, a battery 102 and an A/D (Analog-Digital) module 103. The photoelectric sensor 2 includes a photoelectric heart rate sensor 21, a photoelectric blood oxygen sensor 22, and a blood pressure sensor 23. The photoelectric heart rate sensor 21, the photoelectric blood oxygen sensor 22, the blood pressure sensor 23, the motion sensor 3, the body temperature sensor 4 and the GPS sensor 5 are all electrically coupled to the A/D converting module 103. The A/D converting module 103 is electrically coupled to the monitor MCU 6. The monitor MCU 6 is electrically coupled to the network camera 7 through the wireless transmitting module 101. The network camera 7 includes a Wi-Fi module 71, a mobile communication module 72, a RJ45 module 73, a data receiving module 74, a camera MCU 75 and an A/V module 76. The data receiving module 74 is electrically coupled to the wireless transmitting module 101. The data receiving module 74 and the A/V module 76 are electrically coupled to the camera MCU 75. The camera MCU 75 is electrically coupled to the router 8 through the Wi-Fi module 71 or the RJ45 module 73. The router 8 is electrically coupled to the server 9. The network camera 7 can directly transmit the physiological characteristics signal and the audio and video signal to the server 9 through the mobile communication module 72. The server 9 can push the physiological characteristics signal and the audio and video signal to the terminal device 10. The terminal device 10 is electrically coupled to the server 9 through a uniform communicating protocol. The mobile communication module 72 can be a 3G/4G/5G/6G communication module.

At least two of the vital signs monitor 1, the network camera 7, the router 8, the server 9 and the terminal device 10 have separate communicating protocol. The motion sensor 3 is a three-axis acceleration sensor, which can measure acceleration along X-axis, Y-axis and Z-axis to detect motion of user. The vital signs monitor 1 is communicated with the network camera 7 through a wireless communication protocol, selected from but not limited to one of the Bluetooth, the Microwave, the Zigbee, and the Wi-Fi. The network camera 7 and the router 8 are connected through the Wi-Fi or the RJ54 network. The camera MCU 75 includes a codec to transmit data received by the data receiving module 74 and the A/V module 76. The vital signs monitor 1 can be a wrist band, an ankle band, a necklet or other wearable devices. The terminal device 10 is a smart phone or a band. The smart phone can connect to the monitor device and receive the physiological information with audio and video data of the user in time.

In use, the vital signs monitor 1 is strapped on a wrist or an ankle of a user. The vital signs monitor 1 can detect physiological characteristics of the user, including heart rate, oxygen content of blood, blood pressure, quantity of motion, body temperature and location of the user. The vital signs monitor 1 can also determine sleep state and sleep quality of the user through the physiological characteristics. The vital signs monitor 1 can receive an analog signal of the physiological characteristics through the physiological characteristics detecting sensors and can transform the analog signal to a digital signal through the A/D converting module 103. The monitor MCU 6 receives and transfers the digital signal to the network camera 7 through the wireless transmitting module 101. The camera MCU 75 of the network camera 7 encodes and decodes the digital signal to a physiological characteristics signal, and transmits the physiological characteristics signal and the audio and video signal captured by the network camera 7 synchronously to the server 9 through the router 8. The network camera 7 can directly transmit the physiological characteristics signal and the audio and video signal to the server 9 through the mobile communication module 72 including 3G/4G/5G/6G communication. The server 9 can push the physiological characteristics signal and the audio and video signal to the terminal device 10. The terminal device 10 is electrically coupled to the server 9 through a uniform communicating protocol. The terminal device 10 can be a smart phone or a smart wrist band to remotely monitor the user.

The monitoring device of the embodiment can transmit the physiological characteristics signal and the audio and video signal synchronously to the remote terminal device. It is safe to the special users, such as the babies or the elders. The camera can also monitor the action of the guardians for the users.

The embodiments shown and described above are only examples. Many details are often found in the art such as the other features of a system for generating picture thumbnail. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure up to, and including the full extent established by the broad general meaning of the terms used in the claims. It will therefore be appreciated that the embodiments described above may be modified within the scope of the claims.

What is claimed as new and desired to be protected by Letters Patent, is:

1. An audio and video monitoring device for detecting physiological information which can be controlled by a terminal device, comprising:
a vital signs monitor, comprising a photoelectric sensor, a motion sensor, a body temperature sensor, a GPS sensor, a monitor MCU, a wireless transmitting module, a battery, and an A/D converting module; the photoelectric sensor comprising a photoelectric heart rate sensor, a photoelectric blood oxygen sensor, and a blood pressure sensor; the photoelectric heart rate sensor, the photoelectric blood oxygen sensor, the blood pressure sensor, the motion sensor, the body temperature sensor and the GPS sensor coupled to the A/D converting module, the A/D converting module coupled to the monitor MCU, the monitor MCU wirelessly connected to the network camera; and a network camera, comprising a Wi-Fi module, a mobile communication module, a RJ45 module, a data receiving module, a camera MCU and an A/V module, the data receiving module coupled to the wireless transmitting module of the vital signs monitor, the data receiving module and the A/V module coupled to the camera MCU, the camera MCU coupled to the router through the Wi-Fi module or the RJ45 module, the router coupled to the server; the camera MCU coupled to the server through the router or the mobile communication module, and the server coupled to the terminal device,
wherein the monitor MCU is electrically coupled to the network camera through the wireless transmitting module.

2. The audio and video monitoring device of claim 1, wherein at least two of the vital signs monitor, the network camera, the router, the server and the terminal device have separate communicating protocol.

3. The audio and video monitoring device of claim 1, wherein the motion sensor is a three-axis acceleration sensor.

4. The audio and video monitoring device of claim 1, wherein the vital signs monitor and the network camera are wirelessly connected, and a communication protocol of the vital signs monitor and the network camera is selected from but not limited to the Bluetooth, the Microwave, the Zigbee, or the WI-FI.

5. The audio and video monitoring device of claim 1, wherein the camera MCU comprises a codec that could re-code data from the vital signs monitor and the A/V signal, and could send the data to a smart phone.

6. The audio and video monitoring device of claim 1, wherein the vital signs monitor is selected from a wrist band, an ankle band and a necklet, and the terminal device is a smart phone or a band.
